(19) United States
(12) Patent Application Publication
(10) Pub. No.: US 2007/0055237 A1
(43) Pub. Date: Mar. 8, 2007

(54) Percutaneous spinal implants and methods

(76) Inventors: Avram Allan Edidin, Sunnyvale, CA (US); Andrew C. Kohm, Burlingame, CA (US); Hugues F. Malandain, Mountain View, CA (US)

Correspondence Address: COOLEY GODWARD KRONISH LLP;ATTN: PATENT GROUP, THE BOWEN BUILDING, 875 15TH STREET, N.W. SUITE 800, WASHINGTON, DC, 20005-2221, US

(21) Appl. No.: 11/356,294

(22) Filed: Feb. 17, 2006

Related U.S. Application Data
(63) Continuation-in-part of application No. 11/252,879, filed on Oct. 19, 2005.
(63) Continuation-in-part of application No. 11/252,880, filed on Oct. 19, 2005.
Application No. 11/252,880 is a continuation-in-part of application No. 11/059,526, filed on Feb. 17, 2005.
(60) Provisional application No. 60/695,836, filed on Jul. 1, 2005.

(51) Int. Cl. A61F 2/30

(52) U.S. Cl. 606/61

(57) ABSTRACT

Apparatuses and methods for performing minimally-invasive medical procedures are described. In one embodiment, for example, an apparatus includes an elongate member having a proximal portion and a distal portion are each configured to move from a first configuration to a second configuration and from the second configuration to the first configuration. At least a section of each of the proximal portion and the distal portion is collapsed in the first configuration and is expanded in the second configuration. A central portion is positioned between the proximal portion and the distal portion. The non-expanding central portion is configured to be disposed between adjacent spinous processes upon spinal extension. A material of the non-expanding central portion is different than a material of the proximal portion and the distal portion.

### CROSS-REFERENCE TO RELATED APPLICATIONS

This application is a continuation-in-part of U.S. patent application Ser. No. 11/252,879, entitled “Percutaneous Spinal Implants and Methods,” filed Oct. 19, 2005; and U.S. patent application Ser. No. 11/252,880, entitled “Percutaneous Spinal Implants and Methods,” filed Oct. 19, 2005, each of which is a continuation-in-part of U.S. patent application Ser. No. 11/059,526, entitled “Apparatus and Method for Treatment of Spinal Conditions,” filed Feb. 17, 2005 and claims the benefit of U.S. Provisional Application Ser. No. 60/695,836 entitled “Percutaneous Spinal Implants and Methods,” filed Jul. 1, 2005, each of which is incorporated herein by reference in its entirety.

This application is related to U.S. patent application Attorney Docket Nos. KYPH-001/04US, KYPH-001/05US, KYPH-001/06US, and KYPH-001/07US each entitled “Percutaneous Spinal Implants and Methods,” and filed on even date herewith, each of which is incorporated herein by reference in its entirety.

### BACKGROUND

The invention relates generally to percutaneous spinal implants, and more particularly, to percutaneous spinal implants for implantation, for example, between adjacent spinous processes and optional subsequent removal therefrom.

A back condition that impacts many individuals is spinal stenosis. Spinal stenosis is a progressive narrowing of the spinal canal that causes compression of the spinal cord. Each vertebra in the spinal column has an opening that extends through it. The openings are aligned vertically to form the spinal canal. The spinal cord runs through the spinal canal. As the spinal canal narrows, the spinal cord and nerve roots extending from the spinal cord and between adjacent vertebrae are compressed and may become inflamed. Spinal stenosis can cause pain, weakness, numbness, burning sensations, tingling, and in particularly severe cases, may cause loss of bladder or bowel function, or paralysis. The legs, calves and buttocks are most commonly affected by spinal stenosis, however, the shoulders and arms may also be affected.

Mild cases of spinal stenosis may be treated with rest or restricted activity, non-steroidal anti-inflammatory drugs (e.g., aspirin), corticosteroid injections (epidural steroids), and/or physical therapy. Some patients find that bending forward, sitting or lying down may help relieve the pain. In some cases, bending forward may create more vertebral space, which may temporarily relieve nerve compression. Because spinal stenosis is a progressive disease, the source of pressure is often corrected surgically (e.g., decompressive laminectomy) when the patient has increasing pain over time. Known surgical procedures can remove bone and other tissues that have impinged upon the spinal canal or put pressure on the spinal cord. For example, two adjacent vertebrae can also be fused during the surgical procedure to prevent an area of instability, improper alignment or slippage, such as that caused by spondylolisthesis. Alternatively, decompression can relieve pressure on the spinal cord or spinal nerve by widening the spinal canal to define more space. This procedure requires that the patient be given a general anesthesia because an incision is made in the patient to access the spine to remove the areas that are contributing to the pressure. This procedure, however, may result in blood loss and an increased chance of significant complications, and usually results in an extended hospital stay.

Minimally invasive procedures have been developed to provide access to the space between adjacent spinous processes such that major surgery is not required.

Known medical devices have been developed to be permanently implanted between spinous processes. Such devices, however, can be subject to wear or can cause collateral conditions that would necessitate the removal of the medical device. The removal of the medical device can be difficult to accomplish percutaneously.

Thus, a need exists for improvements in devices and methods for the treatment of spinal conditions such as spinal stenosis.

### SUMMARY OF THE INVENTION

Apparatuses and methods for performing minimally-invasive medical procedures are described. In one embodiment, for example, an apparatus includes an elongate member having a proximal portion configured to move from a first configuration to a second configuration and from the second configuration to the first configuration. At least a section of the proximal portion is collapsed in the first configuration and is expanded in the second configuration. A distal portion is configured to move from a first configuration to a second configuration and from the second configuration to the first configuration. At least a section of the distal portion is collapsed in the first configuration and is expanded in the second configuration. A non-expanding central portion is positioned between the proximal portion and the distal portion. The non-expanding central portion is configured to be disposed between adjacent spinous processes upon spinal extension. A material of the non-expanding central portion being different than a material of the proximal portion and the distal portion.

### BRIEF DESCRIPTION OF THE DRAWINGS

FIG. 1 is a schematic illustration of a posterior view of a medical device according to an embodiment of the invention in a first configuration adjacent two adjacent spinous processes.

FIG. 2 is a schematic illustration of a posterior view of a medical device according to an embodiment of the invention in a second configuration adjacent two adjacent spinous processes.

FIG. 3 is a schematic illustration of a deforming element according to an embodiment of the invention in a first configuration.

FIG. 4 is a schematic illustration of a side view of the expanding element illustrated in FIG. 3.

FIG. 5 is a side cross-sectional view of a medical device according to an embodiment of the invention in a first configuration.

FIG. 6 is a side cross-sectional view of the medical device illustrated in FIG. 5 in a second configuration.

FIG. 7 is a cross-sectional side view of a medical device and an actuator according to an embodiment of the invention with a portion of the medical device deployed in a second configuration.

FIG. 8 is a side cross-sectional view of a medical device and an actuator according to an embodiment of the invention with the medical device fully deployed in the second configuration.

FIG. 9 is a side cross-sectional view of a medical device according to another embodiment of the invention in a first configuration.

FIG. 10 is a side cross-sectional view of the medical device illustrated in FIG. 9 in a second configuration.

FIG. 11 is a side cross-sectional view of a medical device and an actuator according to an embodiment of the invention with a portion of the medical device moved back to its first configuration.

FIG. 12 is a side cross-sectional view of a medical device and an actuator according to an embodiment of the invention with the medical device moved back to its first configuration.

FIG. 13 is a side cross-sectional view of a medical device and an actuator according to an embodiment of the invention with a portion of the medical device moved back to its first configuration.

FIG. 14 is a side cross-sectional view of a medical device and an actuator according to an embodiment of the invention with the medical device moved back to its first configuration.

### DETAILED DESCRIPTION

As used in this specification and the appended claims, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the implant end first inserted inside the patient's body would be the distal end of the implant, while the implant end to last enter the patient's body would be the proximal end of the implant.

An apparatus includes an elongate member having a proximal portion configured to be repeatedly moved between a first configuration and a second configuration under, for example, an axial load or a radial load. The elongate member has a distal portion configured to be moved from a first configuration to a second configuration under, for example, an axial load or a radial load. A non-expanding central portion is positioned between the proximal portion and the distal portion. The non-expanding central portion is configured to engage adjacent spinous processes upon spinal extension.

In some embodiments, the elongate member can have multiple portions that each move from a first configuration to a second configuration, either simultaneously or serially. Additionally, the device, or portions thereof, can be configured into many intermediate positions during the movement between the first configuration and the second configuration. For ease of reference, the entire device is referred to as being in either a first configuration or a second configuration although it should be understood that the device and/or portions thereof have a range of motion that includes many configuration including the first configuration and the second configuration.

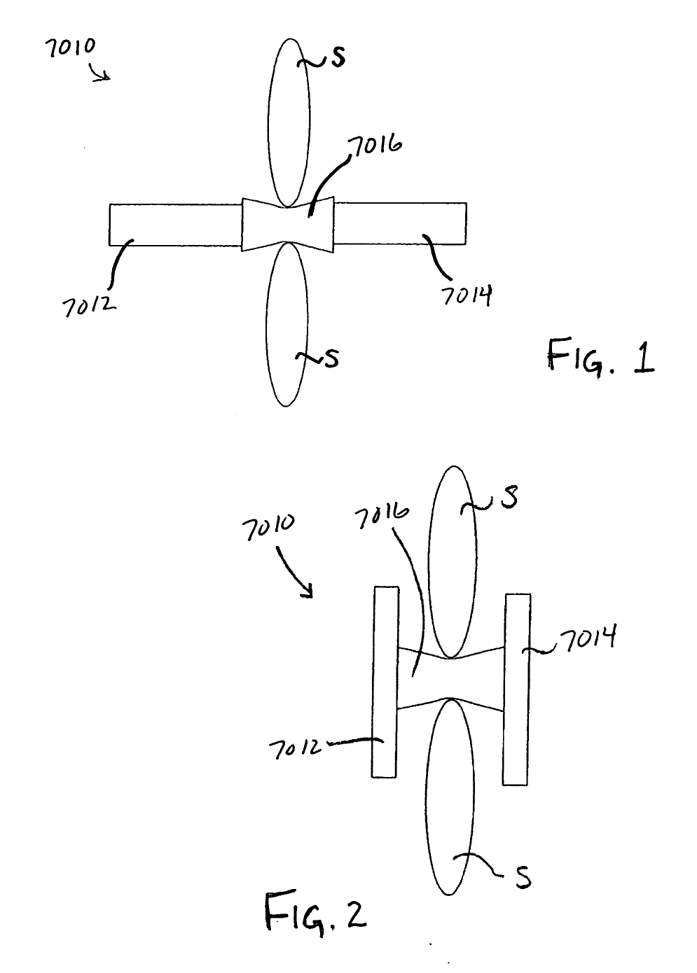

FIG. 1 is a schematic illustration of a medical device according to an embodiment of the invention adjacent two adjacent spinous processes. The medical device 7010 includes a proximal portion 7012, a distal portion 7014 and a central portion 7016. The medical device 7010 has a first configuration in which it can be inserted between adjacent spinous processes S or removed from between adjacent spinous processes S. The central portion 7016 is configured to contact the spinous processes S to prevent over-extension/compression of the spinous processes S. In some embodiments, the central portion 7016 does not substantially distract the adjacent spinous processes S. In other embodiments, the central portion 7016 does not distract the adjacent spinous processes S. The medical device 7010 is inserted into a patient's back and moved in between adjacent spinous processes from the side of the spinous processes (i.e., a posterior-lateral approach). The use of a curved insertion shaft assists in the use of a lateral approach to the spinous processes S.

In the first configuration, the proximal portion 7012, the distal portion 7014 and the central portion 7016 share a common longitudinal axis. In other embodiments, these portions do not share a common longitudinal axis. In some embodiments, the proximal portion 7012, the distal portion 7014 and the central portion 7016 define a tube having a constant inner diameter. In other embodiments, the proximal portion 7012, the distal portion 7014 and the central portion 7016 define a tube having a constant outer diameter and/or inner diameter. In yet other embodiments, the proximal portion 7012, the distal portion 7014 and/or the central portion 7016 have different inner diameters and/or outer diameters.

The medical device 7010 can be moved from the first configuration to a second configuration as illustrated in FIG. 2. In the second configuration, the proximal portion 7012 and the distal portion 7014 are positioned to limit lateral movement of the device 7010 with respect to the spinous processes S. The proximal portion 7012 and the distal portion 7014 are configured to engage the spinous process (i.e., either directly or through surrounding tissue) in the second configuration. For purposes of clarity, the tissue surrounding the spinous processes S is not illustrated. Note the medical device and/or its portions can engage the spinous processes S during all or just a portion of the range of motion of the spinous processes S associated with the patient's movements.

In some embodiments, the proximal portion 7012, the distal portion 7014 and the central portion 7016 are monolithically formed. In other embodiments, one or more of the proximal portion 7012, the distal portion 7014 and the central portion 7016 are separate components that can be coupled together to form the medical device 7010. For example, the proximal portion 7012 and distal portion 7014 can be monolithically formed and the central portion 7016 can be a separate component that is coupled thereto. The proximal portion 7012, the distal portion 7014 and the central portion 7016 can be the same or different materials. These various portions can be coupled, for example, by a friction fit, welding, adhesive, etc.

In use, the spinous processes S can be distracted prior to inserting the medical device 7010. Distraction of spinous processes is disclosed, for example, in U.S. application Ser. No. 11/059,526, incorporated herein by reference in its entirety. When the spinous processes are distracted, a trocar can be used to define an access passage for the medical device 7010. In some embodiments, the trocar can be used to define the passage as well as distract the spinous processes S. Once an access passage is defined, the medical device 7010 is inserted percutaneously and advanced between the spinous processes, distal end 7014 first, until the central portion 7016 is located between the spinous processes S. Once the medical device 7010 is in place between the spinous processes, the proximal portion 7012 and the distal portion 7014 are moved to the second configuration, either serially or simultaneously.

In some embodiments, the medical device 7010 is inserted percutaneously (i.e., through an opening in the skin) and in a minimally-invasive manner. For example, as discussed in detail herein, when inserted, the sizes of portions of the implant are smaller than the size of the opening. The sizes of portions of the implant are expanded after the implant is inserted between the spinous processes. Once expanded, the sizes of the expanded portions of the implant are greater than the size of the opening. When collapsed, the sizes of portions of the spinal implant are again smaller than the size of the opening. For example, the size of the opening/incision in the skin can be between 3 millimeters in length and 25 millimeters in length across the opening. In some embodiments, the size of the implant in the expanded configuration is between 3 and 25 millimeters across the opening.

In some embodiments, the proximal portion 7012 and the distal portion 7014 can be moved back to their original configuration or substantially close to their original configuration and either repositioned between the adjacent spinous processes or removed from the body in which they were inserted.

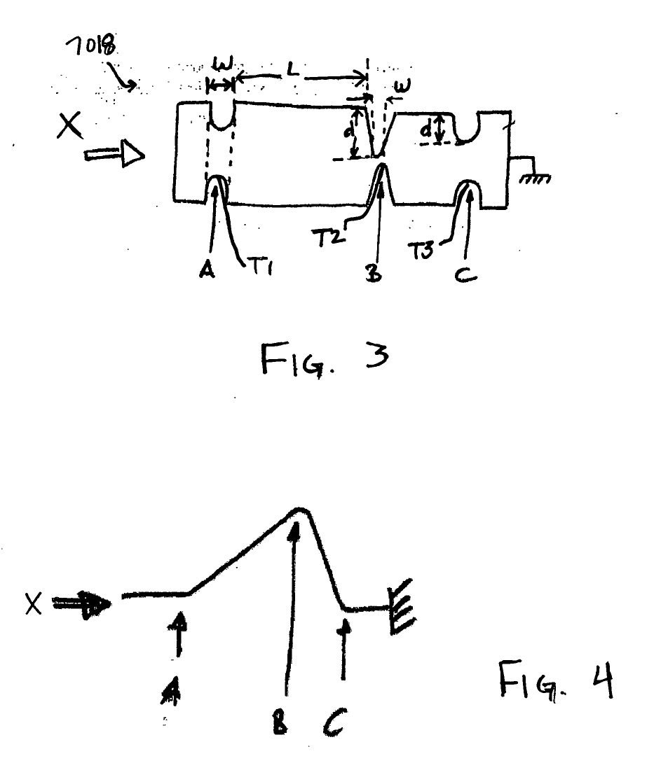

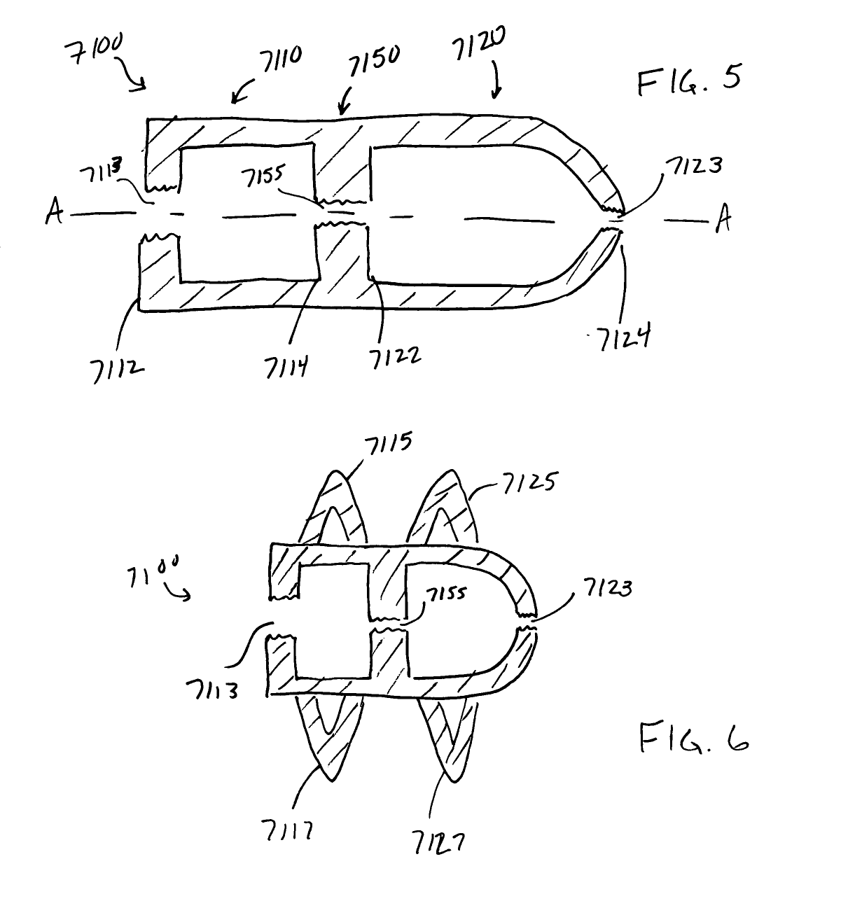

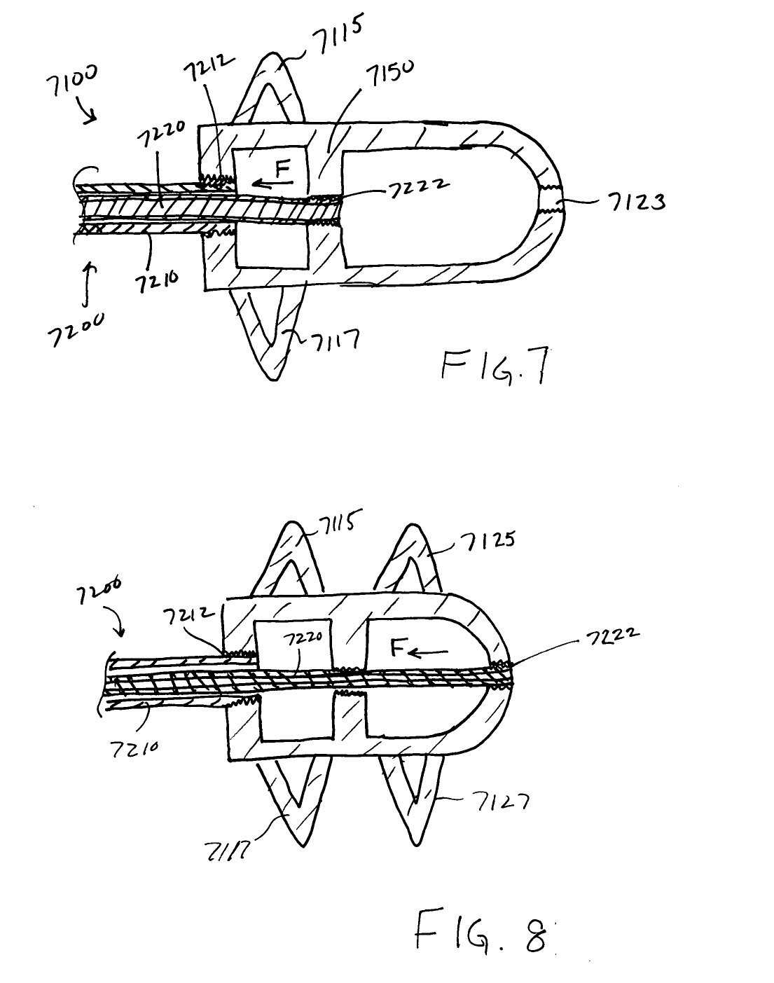

FIG. 3 is a schematic illustration of a deformable element 7018 that is representative of the characteristics of, for example, the distal portion 7014 of the medical device 7010 in a first configuration. The deformable member 7018 includes cutouts A, B, C along its length to define weak points that allow the deformable member 7018 to deform in a predetermined manner. Depending upon the depth d of the cutouts A, B, C and the width w of the throats T1, T2, T3, the manner in which the deformable member 7018 deforms under an applied load can be controlled and varied. Additionally, depending upon the length L between the cutouts A, B, C (i.e., the length of the material between the cutouts), the manner in which the deformable member 7018 deforms can be controlled and varied.

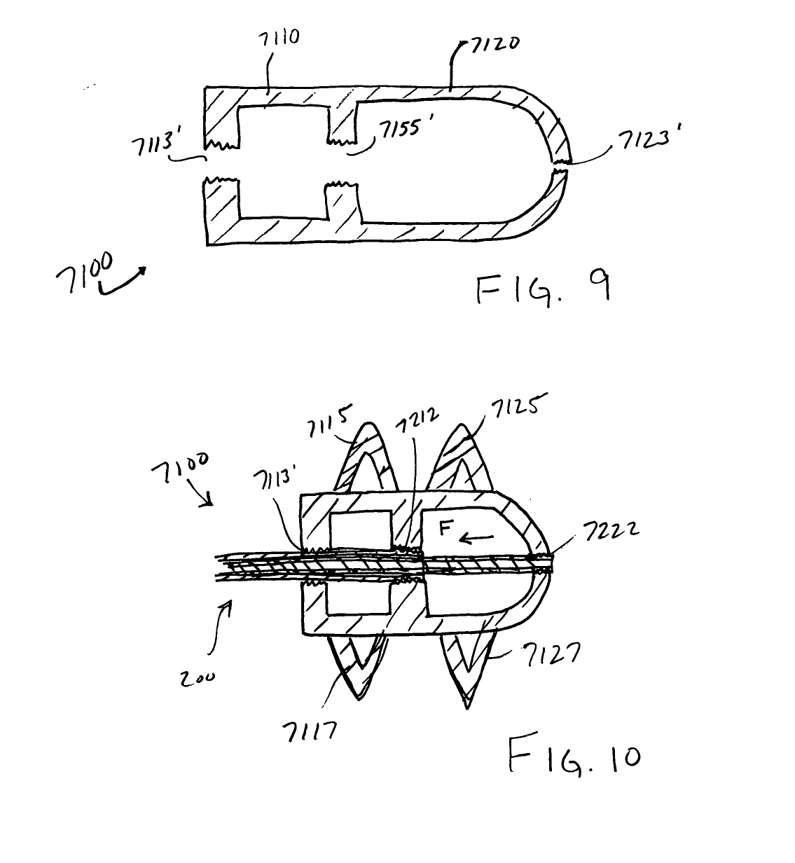

FIG. 4 is a schematic illustration of the expansion properties of the deformable member 7018 illustrated in FIG. 3. When a load is applied, for example, in the direction indicated by arrow X, the deformable member 7018 deforms in a predetermined manner based on the characteristics of the deformable member 7018 as described above. As illustrated in FIG. 4, the deformable member 7018 deforms most at cutouts B and C due to the configuration of the cutout C and the short distance between cutouts B and C. In some embodiments, the length of the deformable member 7018 between cutouts B and C is sized to fit one side of adjacent spinous processes.

The deformable member 7018 is stiffer at cutout A due to the shallow depth of cutout A. As indicated in FIG. 4, a smooth transition is defined by the deformable member 7018 between cutouts A and B. Such a smooth transition causes less stress on the tissue surrounding a side of adjacent spinous processes than a more drastic transition (i.e., a steeper angled wall) such as between cutouts B and C. The dimensions and configuration of the deformable member 7018 can also determine the timing of the deformation at the various cutouts. The weaker (i.e., deeper and wider) cutouts deform before the stronger (i.e., shallower and narrower) cutouts.

FIGS. 5 and 6 illustrate a spinal implant 7100 in a first configuration and second configuration, respectively. As shown in FIG. 5, the spinal implant 7100 is collapsed in a first configuration and can be inserted between adjacent spinous processes. The spinal implant 7100 has a first deformable portion 7110, a second deformable portion 7120 and a central, non-deformable portion 7150. The first deformable portion 7110 has a first end 7112 and a second end 7114. The second deformable portion 7120 has a first end 7122 and a second end 7124. The central portion 7150 is coupled between second end 7114 and first end 7122. In some embodiments, the spinal implant 7100 is monolithically formed.

The first deformable portion 7110, the second deformable portion 7120 and the central portion 7150 have a common longitudinal axis A along the length of spinal implant 7100. The central portion 7150 can have the same inner diameter as first deformable portion 7110 and the second deformable portion 7120. In some embodiments, the outer diameter of the central portion 7150 is smaller than the outer diameter of the first deformable portion 7110 and the second deformable portion 7120.

In use, spinal implant 7100 is inserted percutaneously between adjacent spinous processes. The first deformable portion 7110 is inserted first and is moved past the spinous processes until the central portion 7150 is positioned between the spinous processes. The outer diameter of the central portion 7150 can be slightly smaller than the space between the spinous processes to account for surrounding ligaments and tissue. In some embodiments, the central portion 7150 directly contacts the spinous processes between which it is positioned. In some embodiments, the central portion of spinal implant 7100 is a fixed size and is not compressible or expandable. Note the spinal implant 7100 and/or the first deformable portion 7110, second deformable portion 7120, and central portion 7150 can engage the spinous processes during all or just a portion of the range of motion of the spinous processes associated with the patient's movement.

The first deformable portion 7110 includes, for example, expanding members 7115, and 7117. Between the expanding members 7115, 7117, openings (not illustrated) are defined. As discussed above, the size and shape of the openings influence the manner in which the expanding members 7115, 7117 deform when an axial load is applied. The second deformable portion 7120 includes expanding members 7125 and 7127. Between the expanding members 7125, 7127, openings (not illustrated) are defined. As discussed above, the sizes and shapes of the openings influence the manner in which the expanding members 7125, 7127 deform when an axial load is applied.

When an axial load is applied to the spinal implant 7100, the spinal implant 7100 expands to a second configuration as illustrated in FIG. 6. In the second configuration, first end 7112 and second end 7114 of the first deformable portion 7110 move towards each other and expanding members 7115, 7117 project substantially laterally away from the longitudinal axis A. Likewise, first end 7122 and second end 7124 of the second deformable portion 7120 move towards one another and expanding members 7125, 7127 project laterally away from the longitudinal axis A. The expanding members 7115, 7117, 7125, 7127 in the second configuration form projections that extend to positions adjacent to the spinous processes between which the spinal implant 7100 is inserted. In the second configuration, the expanding members 7115, 7117, 7125, 7127 inhibit lateral movement of the spinal implant 7100, while the central portion 7150 prevents the adjacent spinous processes from moving together any closer than the distance defined by the diameter of the central portion 7150 during spinal extension.

The first end 7112 of the first deformable portion 7110 defines a threaded opening 7113. The central portion 7150 defines a second threaded opening 7155. The second end 7124 of the second deformable portion 7120 defines a third threaded opening 7123. The threaded openings 7113, 7155, 7123 receive portions of an actuator 7200 (see FIG. 7) to move the first deformable portion 7100 and the second deformable portion 7120 between their respective first configurations and second configurations as described in greater detail herein. In some embodiments, the first threaded opening 7113 has a greater diameter than the second threaded opening 7155 and the third threaded opening 7123 (see FIGS. 5-8). In some embodiments the second threaded opening 7155 and the third threaded opening 7123 have the same diameter (see FIGS. 5-8). In other embodiments, the first threaded opening 7113′ and the second threaded opening 7155′ have the same diameter (see FIGS. 9-12) and the third threaded opening 7123′ has a smaller diameter than the first threaded opening and the second threaded opening. The threaded openings 7113, 7155, 7123, 7113′, 7155′, 7123′ are coaxially aligned. In other embodiments, the threaded openings can be any combination of different or the same sizes.

The spinal implant 7100 is deformed by a compressive force imparted substantially along the longitudinal axis A of the spinal implant 7100. As illustrated in FIG. 7, the compressive force is imparted to the first deformable portion 7110 by actuator 7200. The actuator includes a first portion 7210 and a second portion 7220 movably received within first portion 7210. In some embodiments, the second portion 7220 is slidably received within the first portion 7210. In other embodiments, the first portion 7210 and the second portion 7220 are threadedly coupled. Each of the first portion 7210 and the second portion 7220 is provided with external threads 7212 and 7222, respectively, to engage the threaded openings 7113, 7155, 7123, 7113′, 7155′, 7123′.

As illustrated in FIG. 7, the compressive force is imparted to the first deformable portion 7110, for example, by attaching the threaded portion 7212 to the first threaded opening 7113, attaching the threaded portion 7222 to the second threaded opening 7155 of the central portion 7150, and drawing the second portion 7220 along the longitudinal axis A while imparting an opposing force against the first end 7112 of the first deformable portion 7110. The opposing force results in a compressive force causing the spinal implant 7100 to expand as discussed above.

Once the first deformable portion 7110 is moved to its second configuration, the threaded portion 7222 is threaded through the second threaded opening 7155 and threadedly coupled to the third threaded opening 7123. A compressive force is imparted to the second deformable portion 7120 of the spinal implant 7100 by drawing the second portion 7220 of the actuator in the direction indicated by the arrow F while applying an opposing force using the first portion 7210 of the actuator against the spinal implant 7100. The opposing forces result in a compressive force causing the spinal implant to expand as illustrated in FIG. 8.

In some embodiments, the first deformable portion 7110 and the second deformable portion 7120 can be expanded simultaneously when the second portion 7220 of the actuator is coupled to the third threaded opening 7123 and the first portion 7210 is coupled to the first threaded opening 7113 and a compressive force is applied.

In embodiments in which the first threaded opening 7113′ has the same diameter as the second threaded opening 7155′ (best seen, for example, in FIGS. 9 and 10), the first threaded portion 7212 can be threadedly coupled to the second threaded opening 7155′ and the second threaded portion 7222 can be threadedly coupled to the third threaded opening 7123′. A compressive force is then applied between the central portion 7150 and the second end 7124 of the second deformable portion 7120. Once the second deformable portion 7120 is in its second configuration, the first threaded portion 7212 can be threadedly coupled to the first threaded opening 7113′ and the first deformable portion 7110 can be deformed into its second configuration.

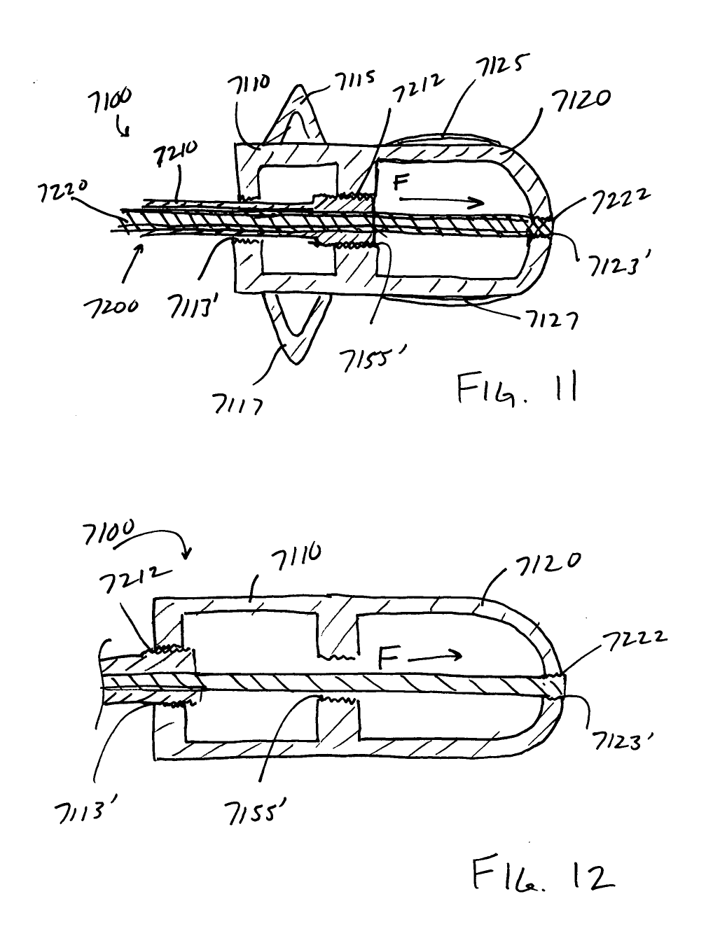

After each of the first deformable portion 7110 and the second deformable portion 7120 are moved to the second expanded configuration, they subsequently can each be moved back to the first collapsed configuration by applying a force in the opposite direction along longitudinal axis A as illustrated, for example, in FIGS. 11-12. In this example, as discussed above, the spinal implant 7100 illustrated in FIGS. 9-12 has a first threaded opening 7113′ that has the same diameter as the second threaded opening 7155′.

With the first threaded portion 7212 coupled to the second threaded opening 7155′ and the second threaded portion 7222 coupled to the third threaded opening 7123′, the second portion 7220 of the actuator 7200 is moved in the direction indicated by arrow F to move the second deformable portion 7120 to its first collapsed configuration.

The first threaded portion 7212 is then coupled to the first threaded opening 7113′ and the second portion 7220 of actuator 7200 is again moved in the direction of arrow F to move the first deformable portion 7110 to its first collapsed configuration. When the entire spinal implant 7100 has been completely collapsed, the spinal implant 7100 can be repositioned between the spinous processes, or removed from its position between the spinous processes and removed from the body in which it was previously inserted. In some embodiments, the first deformable portion 7110 and the second deformable portion 7120 are not completely collapsed, but are instead moved to a configuration between fully expanded and fully collapsed. In this manner the spinal implant 7100 may be repositioned or removed without being completely collapsed.

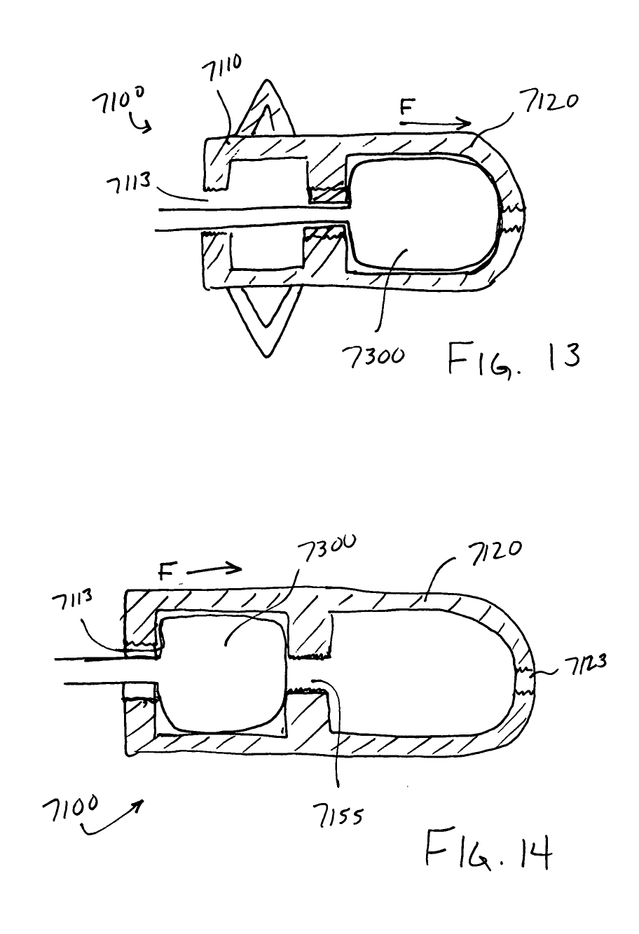

In some embodiments, the first deformable portion 7110 and the second deformable portion 7120 can be moved between the first and second configuration using a balloon as an actuator. As illustrated in FIG. 13, the second deformable portion 7120 is then moved from the second configuration to the first configuration by imparting a longitudinal force resulting from the inflation of a balloon 7300 with liquid and/or gas. As the balloon 7300 is inflated, it is forced against the central portion 7150 and the second end 7124 of the second deformable portion 7120. The force imparted by the balloon 7300 is generally in the direction indicated by the arrow F. In some embodiments, the balloon 7300 is a low-compliant balloon that is configured to expand to a predefined shape such that a force is imparted primarily in a substantially longitudinal direction indicated by arrow F.

After the second deformable portion 7120 is moved substantially to its collapsed configuration, the balloon 7300 is deflated and moved into the first deformable portion 7110. The balloon 7300 is then inflated as illustrated in FIG. 14 to impart a force in the direction indicated by arrow F. In some embodiments, the same balloon 7300 is used to collapse both the first deformable portion 7110 and the second deformable portion 7120. In other embodiments, a different balloon is used for each portion 7110, 7120. Once the entire implant 7100 is moved to the first configuration, the balloon is deflated and removed. In some embodiments, the balloon 7300 remains in the spinal implant 7100, and the spinal implant 7100 and the balloon 7300 are removed simultaneously.

In some embodiments, the shaft on which the balloon is coupled has external threads (not illustrated) to mate with the first threaded opening 7113, 7113′ and/or the second threaded opening 7155, 7155′. In other embodiments, neither the openings nor the shaft on which the balloon is coupled are threaded. In yet other embodiments, the balloon 7300 is inserted through the first portion 7210 of the actuator 7200. Alternatively, the actuator 7200 and the balloon 7300 can be used in conjunction with the spinal implant to expand and/or contract the first deformable portion 7110 and the second deformable portion 7120.

In other embodiments, there are no threaded openings defined in the spinal implant 7100. For example, the spinal implant can have multiple actuator-engaging portions that are not threaded, but are rather contact or bearing surfaces for various types of actuators. For example, an actuator (not illustrated) can be configured to grasp an outer surface of the spinal implant while simultaneously imparting a force against the distal portion of the spinal implant to move the implant to a collapsed configuration.

The spinal implant 7100 can be made from, for example, stainless steel, plastic, polyetheretherketone (PEEK), carbon fiber, ultra-high molecular weight (UHMW) polyethylene, etc. or some combination thereof. For example, the first deformable portion and the second deformable portion can be made from one material and the non-expanding central portion can be made from a different material. The material of such a non-expanding central portion can have a tensile strength similar to or higher than that of bone.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, although the embodiments above are primarily described as being spinal implants configured to be positioned between adjacent spinous processes, in alternative embodiments, the implants are configured to be positioned adjacent any bone, tissue or other bodily structure where it is desirable to maintain spacing while preventing axial or longitudinal movement of the implant.

While the implants described herein were primarily described as not distracting adjacent spinous processes, in alterative embodiments, the implants can be configured to expand to distract adjacent spinous processes.

Although described as being inserted directly between adjacent spinous processes, in alternative embodiments, the implants described above can be delivered through a cannula.

Although the actuator used to move the spinal implant from the expanded configuration to the collapsed configuration is described as a rod assembly or a balloon, in alternative embodiments the actuator can be any device configured to impart a longitudinal force sufficient to move the implant to its collapsed configuration. For example, the actuator can be a piston/cylinder assembly, a ratchet assembly, or the like.

1. An apparatus, comprising:
an elongate member having
a proximal portion configured to move from a first configuration to a second configuration and from the second configuration to the first configuration, at least a section of the proximal portion being collapsed in the first configuration and expanded in the second configuration;
a distal portion configured to move from a first configuration to a second configuration and from the second configuration to the first configuration, at least a section of the distal portion being collapsed in the first configuration and expanded in the second configuration; and
a central portion between the proximal portion and the distal portion, the central portion configured to be disposed between adjacent spinous processes upon spinal extension, a material of the central portion being different than a material of the proximal portion and the distal portion.

2. The apparatus of claim 1, further comprising:
a proximal actuator-engaging portion; and
a distal actuator-engaging portion, the proximal actuator-engaging portion and the distal actuator-engaging portion each being configured to receive at least a portion of an actuator to cause the elongate member to move between the first configuration and the second configuration under an axial load.

3. The apparatus of claim 1, wherein the proximal portion is configured to receive a balloon actuator to cause the proximal portion to move from its second configuration to its first configuration when the balloon actuator is inflated.

4. The apparatus of claim 1, wherein the proximal portion and the distal portion are configured to deform from the second configuration to the first configuration by an axially applied load imparted by an expansion of a low-compliant balloon.

5. The apparatus of claim 1, further comprising:
an actuator having a shaft and an expandable portion coupled to the shaft, the expandable portion configured to expand within the distal portion to cause the distal portion to move from its second configuration to its first configuration.

6. The apparatus of claim 1, further comprising:
an actuator having a shaft and an expandable portion coupled to the shaft, the expandable portion configured to expand within the proximal portion to cause the proximal portion to move from its second configuration to its first configuration.

7. The apparatus of claim 1, the material of the proximal portion and the material of the distal portion are configured to deform in response to an axially applied load imparted by an expansion of a low-compliant balloon.

8. The apparatus of claim 1, wherein the proximal portion and the distal portion are configured to deform from the first configuration to the second configuration under a first axial load applied in a first direction, and the proximal portion and the distal portion are configured to move from the second configuration to the first configuration under a second axial load applied in a second direction, the second direction being substantially opposite the first direction.

9. A method, comprising:
inserting an expandable member into an elongate member, the elongate member including a proximal end portion having a section in an expanded configuration and a distal end portion having a section in an expanded configuration; and
expanding the expandable member in a longitudinal to cause the section of the distal end portion to move from its expanded configuration to a collapsed configuration.

10. The method of claim 9, further comprising:
expanding the expandable member in a longitudinal direction to cause the section of the proximal end portion to move from its expanded configuration to a collapsed configuration.

11. The method of claim 9, further comprising removing the expandable member from the elongate member after at least one of the proximal end portion is in the collapsed configuration and the distal end portion is in the collapsed configuration.

12. The method of claim 9, wherein the expanding the expandable member includes inflating a balloon.

13. The method of claim 9, wherein the expanding the expandable member includes actuating a piston.

14. The method of claim 9, further comprising:
coupling the expandable member to the elongate member such that the expandable member is within the proximal portion before the expanding the expandable member to cause the section of the proximal end portion to move from its expanded configuration to a collapsed configuration;
separating the expandable member from the elongate member after the expanding the expandable member to cause the section of the proximal end portion to move from its expanded configuration to a collapsed configuration; and
coupling the expandable member to the elongate member such that the expandable member is within the distal portion before the expanding the expandable member to cause the section of the distal end portion to move from its expanded configuration to a collapsed configuration.

15. A kit, comprising:
an elongate member having
a proximal portion configured to move from a first configuration to a second configuration and from the second configuration to the first configuration, at least a section of the proximal portion being collapsed in the first configuration and expanded in the second configuration;
a distal portion configured to move from a first configuration to a second configuration and from the second configuration to the first configuration, at least a section of the distal portion being collapsed in the first configuration and expanded in the second configuration; and
a central portion between the proximal portion and the distal portion, the central portion configured to be disposed between adjacent spinous processes upon spinal extension, the central portion being a different material than a material of the proximal portion and the distal portion; and
an actuator configured to move the section of the proximal portion to its first configuration from its second configuration and move the section of the distal portion to its first configuration from its second configuration.

16. The kit of claim 15, wherein the actuator includes a balloon.

17. The kit of claim 15, wherein the actuator includes a piston and cylinder assembly.

18. The kit of claim 15, wherein the actuator is hydraulically actuated.

19. The kit of claim 15, wherein
the proximal portion defines a volume when the proximal portion is in the first configuration, and
the actuator is configured to define a volume when the actuator is in a collapsed configuration and define a volume when the actuator is in an expanded configuration, the volume of the actuator when in its expanded configuration substantially corresponding to the volume of the proximal portion when in its first configuration.

20. The kit of claim 15, wherein
the distal portion defines a volume when the distal portion is in the first configuration, and
the actuator is configured to define a volume when the actuator is in a collapsed configuration and define a volume when the actuator is in an expanded configuration, the volume of the actuator when in its expanded configuration substantially corresponding to the volume of the distal portion when in its first configuration.

21. The kit of claim 15, wherein the actuator is configured to be inserted in the elongate member subsequent to placement of the elongate member between adjacent spinous processes.

22. The kit of claim 15, wherein the actuator is configured to removably engage the elongate member.
